(19) United States
(12) Patent Application Publication
(10) Pub. No.: US 2026/0207804 A1
(43) Pub. Date: Jul. 23, 2026

(54) AIR CONDITIONER CLEANING DEVICE

(71) Applicant: Michael Ojeda, Spring, TX (US)

(72) Inventor: Michael Ojeda, Spring, TX (US)

(21) Appl. No.: 19/034,010

(22) Filed: Jan. 22, 2025

(51) Int. Cl. A61L 2/18; A61L 2/24; F24F 13/00

(52) U.S. Cl. CPC A61L 2/18; A61L 2/24; F24F 13/00; A61L 2103/23; A61L 2202/14; A61L 2202/15; F24F 2221/225

(57) ABSTRACT

An air conditioner cleaning device includes an air handler of a heating, ventilation and air conditioning system. The air handler has an evaporator coil and a drain pan to capture condensation that drips from the evaporator coil. A housing is mountable to the air handle and a pump is integrated into the housing. A container contains a liquid cleaner and an input of the pump is in fluid communication with the container. An output of the pump is in fluid communication with the drain pan to deliver the liquid cleaner into the drain pan when the pump is turned for cleaning and sanitizing the condensation that is captured in the drain pan such that mold and algae are inhibited from growing in the condensation which could potentially obstruct a drain line of the drain pan.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to air conditioner devices and more particularly pertains to a new air conditioner device for sanitizing condensation from an evaporator coil that collects in a drain pan. The device includes a housing that is mountable to an air handler of a heating, ventilation and air conditioning system and a pump that is positioned within the housing. The device includes a pump positioned within the housing that has an input and an output. The input is in fluid communication with a container which contains a liquid cleaner and the output is in fluid communication with the drain pan. The pump is programmable to turn on and off at predetermined times for a predetermined duration of time to sanitize the condensation in the drain pan to inhibit the growth of mold and algae.

### (2) Description of Related Art Including Information Disclosed Under 37 CFR 1.97 and 1.98

The prior art relates to air conditioner devices including: an aerosol duct treatment apparatus that regularly sprays an aerosol sanitizer into ducts of an air conditioning system to sanitize the ducts; a variety of sanitizer dispenser devices that each at least includes a pump that is in fluid communication with a container of a liquid cleaner to deliver the liquid cleaner directly into a condensate drain line to inhibit the growth of mold or algae in the drain line. In no instance does the prior art disclose an air conditioning cleaning device that includes a pump that is mountable to an air handler that is in fluid communication with a container of a liquid cleaner to deliver the liquid cleaner into a drain pan of an evaporator coil of the air handler to inhibit the growth of mold or algae in the drain pan thereby reducing the likelihood of clogs developing in a drain line of the drain pan.

### BRIEF SUMMARY OF THE INVENTION

An embodiment of the disclosure meets the needs presented above by generally comprising an air handler of a heating, ventilation and air conditioning system. The air handler has an evaporator coil and a drain pan to capture condensation that drips from the evaporator coil. A housing is mountable to the air handle and a pump is integrated into the housing. A container contains a liquid cleaner and an input of the pump is in fluid communication with the container. An output of the pump is in fluid communication with the drain pan to deliver the liquid cleaner into the drain pan when the pump is turned for cleaning and sanitizing the condensation that is captured in the drain pan such that mold and algae are inhibited from growing in the condensation which could potentially obstruct a drain line of the drain pan.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an air conditioner cleaning device according to an embodiment of the disclosure.

FIG. 2 is a front phantom view of a housing of an embodiment of the disclosure.

FIG. 3 is a perspective view of a container and a housing of an embodiment of the disclosure.

FIG. 4 is a front view of a display of an embodiment of the disclosure showing a menu being displayed on the display.

FIG. 5 is a schematic view of an embodiment of the disclosure.

FIG. 6 is a left side view of a housing of an embodiment of the disclosure.

### DETAILED DESCRIPTION OF THE INVENTION

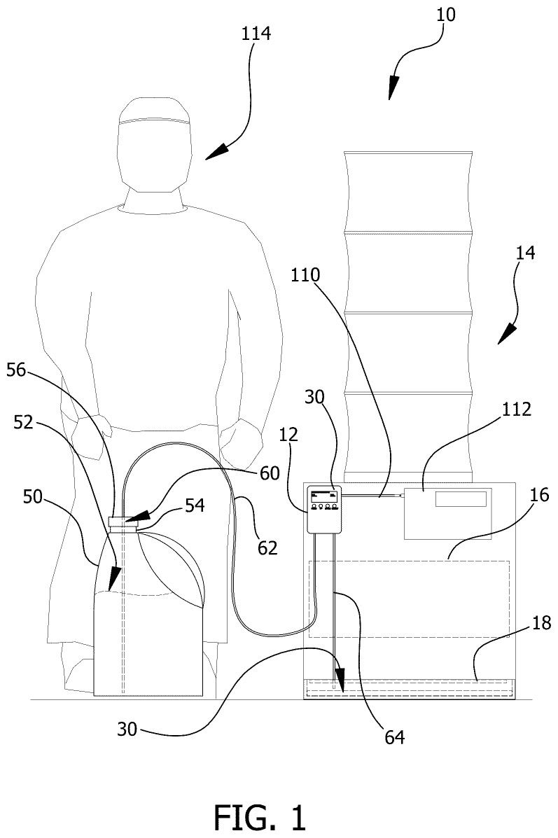

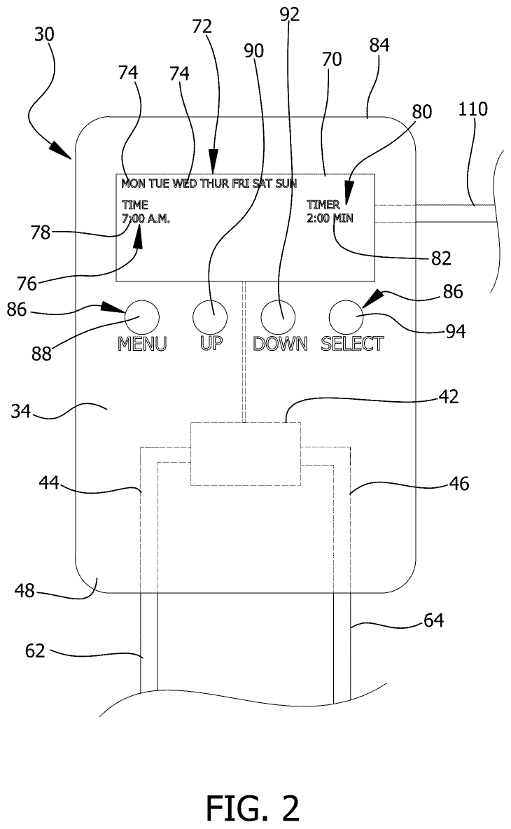

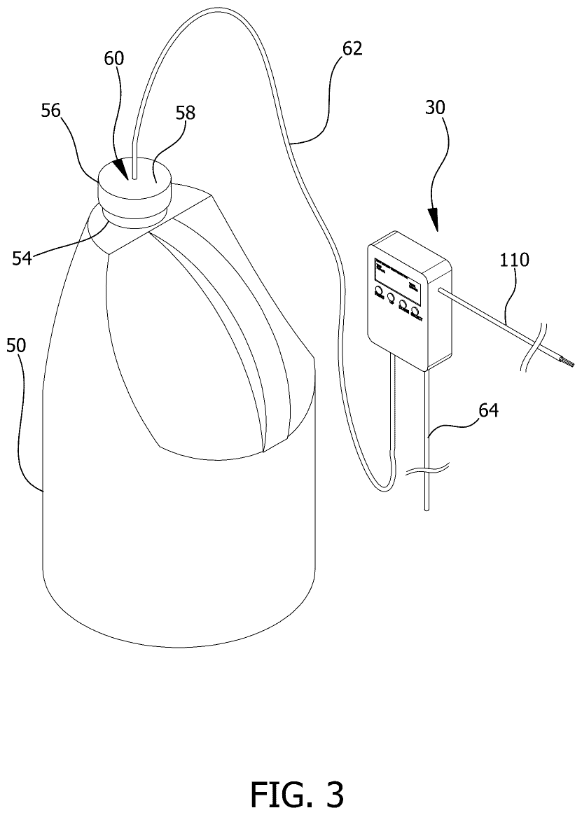

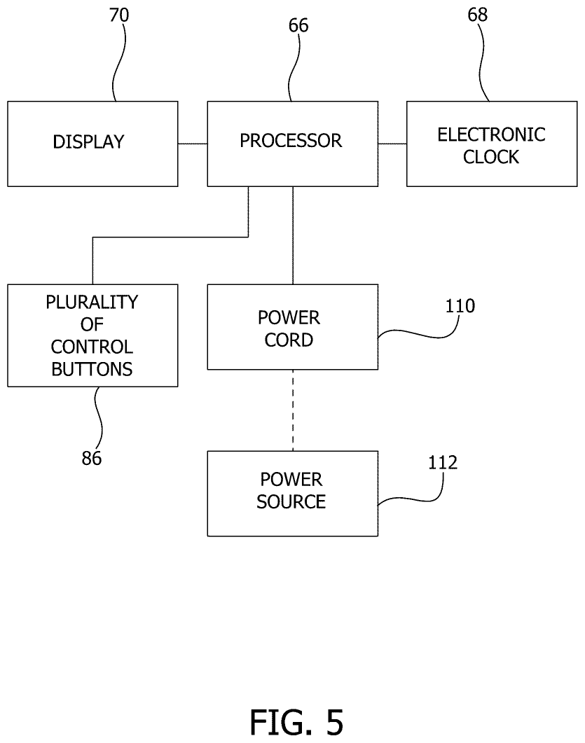

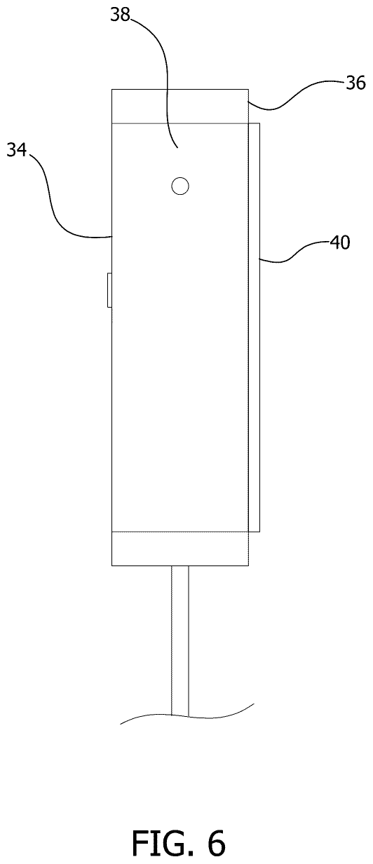

With reference now to the drawings, and in particular to FIGS. 1 through 6 thereof, a new air conditioner device embodying the principles and concepts of an embodiment of the disclosure and generally designated by the reference numeral 10 will be described.

As best illustrated in FIGS. 1 through 6, the air conditioner cleaning device 10 generally comprises an air handler 12 of a heating, ventilation and air conditioning system 14. The air handler 12 has an evaporator coil 16 and the evaporator coil 16 has a drain pan 18 to capture condensation 30 that drips from the evaporator coil 16. The heating, ventilation and air conditioning system 14 may be of any conventional design, including but not being limited to a central air system, which is installed in a residential structure. A housing 32 is mountable to the air handler 12 and the housing 32 has a front wall 34 and a back wall 36 and an outer wall 38 extending between the front wall 34 and the back wall 36. The housing 32 has an attachment element 40 which is disposed on the back wall 36 and the attachment element 40 is attachable to the air handler 12. The attachment element 40 may comprise an adhesive pad which adheres to the air handler 12 or a magnet which magnetically engages the air handler 12 or any other type of attachment element 40 that can retain the housing 32 on the air handler 12.

A pump 42 is provided and the pump 42 is positioned within the housing 32; the pump 42 has an input 44 and an output 46. The outer wall 38 of the housing 32 has a bottom side 48; the input 44 extends through the bottom side 48 and the output 46 extends through the bottom side 48. The pump 42 may comprise peristaltic pump or other type of electronic fluid pump. A container 50 is provided which contains a liquid cleaner 52. The container 50 has a neck 54 and the neck 54 is open into an interior of the container 50. Furthermore, the input 44 of the pump 42 is in fluid communication with the container 50 and the pump 42 urges the liquid cleaner 52 into the input 44 and outwardly through the output 46 when the pump 42 is turned on. In addition, the output 46 is in fluid communication with the drain pan 18 and the output 46 delivers the liquid cleaner 52 into the drain pan 18 when the pump 42 is turned on. Furthermore, the liquid cleaner 52 cleans and sanitizes the condensation 30 that is captured in the drain pan 18 such that mold and algae are inhibited from growing in the condensation 30 which could potentially obstruct a drain line of the drain pan 18.

A lid 56 is removably attachable to the neck 54 of the container 50. The lid 56 has an upper wall 58 and the lid 56 has a hole 60 extending through the upper wall 58. An input tube 62 is coupled to the input 44 of the pump 42 and the input tube 62 is extendable through the hole 60 in the lid 56. The input tube 62 extends downwardly into the interior of the container 50 and the input tube 62 is submerged in the liquid cleaner 52. Furthermore, the pump 42 urges the liquid cleaner 52 into the input 44 of the pump 42 when the pump 42 is turned on.

An output tube 64 is coupled to the output 46 of the pump 42 and the output tube 64 receives the liquid cleaner 52 when the pump 42 is turned on. The output tube 64 is extendable into the air handler 12 and the output tube 64 is routed into the drain pan 18. Furthermore, the output tube 64 releases the liquid cleaner 52 into the drain pan 18 when the pump 42 is turned on such that the liquid cleaner 52 disinfects and cleans the condensation 30 that collects in the drain pan 18. The container 50 may have a fluid volume of at least 3.5 liters such that the container 50 can contain a volume of the liquid cleaner 52 that is sufficient to keep the drain pan 18 clean and disinfected for at least one year. Additionally, the liquid cleaner 52 may comprise a solution of copper sulfate or a mixture of white vinegar and sodium bicarbonate or any other type of liquid cleaner 52 that is approved for use in heating, ventilation and air conditioning systems.

A processor 66 is positioned within the housing 32 and the pump 42 is in electrical communication with the pump 42. An electronic clock 68 is positioned within the housing 32 and the electronic clock 68 is in electrical communication with the pump 42. Furthermore, the electronic clock 68 tracks a time of day and the electronic clock 68 is actuatable to count down a duration of time associated with a timer function of the electronic clock 68. The pump 42 is turned on when the electronic clock 68 begins counting down the duration of time associated with the timer function. Conversely, the pump 42 is turned off when the electronic clock 68 completes counting down the duration of time associated with the timer function.

A display 70 is coupled to the front wall 34 of the housing 32 and the display 70 is in electrical communication with the pump 42. The display 70 displays day indicia 72 comprising days of the week 74 and the display 70 displays time indicia 76 comprising numbers to indicate the time of day 78. Additionally, the display 70 displays timer function indicia 80 comprising numbers 82 to indicate the duration of time associated with the timer function of the electronic clock 68. The outer wall 38 of the housing 32 has a top side 84 and the display 70 is positioned closer to the top side 84 than the bottom side 48 of the outer wall 38 of the housing 32. Furthermore, the display 70 may comprise a light emitting diode display or other type of electronic display.

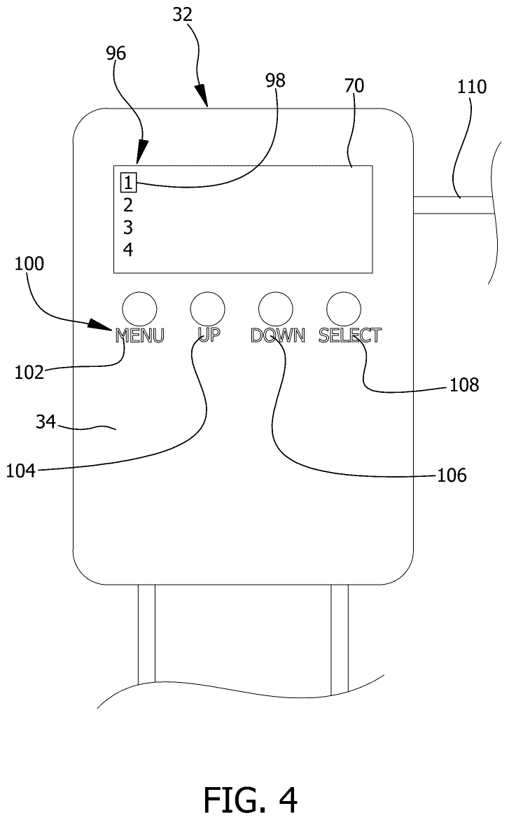

A plurality of control buttons 86 is provided and each control button 86 of the plurality of control buttons 86 is movably integrated into the front wall 34 of the housing 32. Each control button 86 of the plurality of control buttons 86 is in electrical communication with the processor 66. Additionally, the plurality of control buttons 86 includes a menu button 88 and an up button 90 and a down button 92 and a select button 94. The plurality of control buttons 86 is positioned between the display 70 and the bottom side 48 of the outer wall 38 of the housing 32 and the plurality of control buttons 86 is spaced apart from each other and is distributed along an entire width of the display 70. As shown in FIG. 4, the display 70 is actuated to display a menu 96 when the menu button 88 is depressed and the up button 90 moves a cursor 98 upwardly along the menu 96 when the menu 96 is displayed on the display 70 and when the up button 90 is depressed. The down button 92 moves the cursor 98 downwardly along the menu 96 when the menu 96 is displayed on the display 70 and the down button 92 is depressed and the cursor 98 selects an option in the menu 96 when the select button 94 is depressed.

Word indicia 100 are applied to the front wall 34 of the housing 32 and the word indicia 100 comprise the words “menu” 102 and “up” 104 and “down” 106 and “select” 108. The word “menu” 102 is positioned beneath the menu button 88 and the word “up” 104 is positioned beneath the up button 90. Additionally, the word “down” 106 is positioned beneath the down button 92 and the word “select” 108 is positioned beneath the select button 94. A power cord 110 is coupled to and extends away from the outer wall 38 of the housing 32 and the power cord 110 is in electrical communication with the processor 66. The power cord 110 is electrically coupled to a power source 112 comprising a power supply of the air handler 12 such that the power cord 110 can supply electrical power to the processor 66 without requiring a user 114 to replace batteries in the housing 32.

In use, the housing 32 is mounted to the air handler 12, preferably within 1.0 meter of the evaporator coil 16. The container 50 is placed on the ground near the air handler 12 and the input tube 62 is extended through the hole 60 in the lid 56 and the output tube 64 is extended into the air handler 12 such that the output tube 64 can be directed into the drain pan 18 of the evaporator coil 16. A hole 60 can be drilled through the air handler 12 at a strategic location that enables the output tube 64 to be extended through the hole 60 to reach the drain pan 18. The timer function of the electronic clock 68 is programmed in the menu 96 that is displayed on the display 70 to turn on the pump 42 at a specific time of day on a specific day of the week and for a specific duration of time. Furthermore, the pump 42 is automatically turned on and off the keep the condensation 30 in the drain pan 18 clean and sanitized to ensure optimum performance of the drain pan 18 over extended durations of time. The container 50 is refilled with the liquid cleaner 52 when the liquid cleaner 52 has been depleted.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An air conditioner cleaning device comprising:
an air handler of a heating, ventilation and air conditioning system, said air handler having an evaporator coil, said evaporator coil having a drain pan wherein said drain pan is configured to capture condensation that drips from said evaporator coil;
a housing being mountable to said air handler;
a pump being integrated into said housing, said pump having an input and an output;
a container containing a liquid cleaner, said input of said pump being in fluid communication with said container, said pump urging said liquid cleaner into said input and outwardly through said output when said pump is turned on, said output being in fluid communication with said drain pan, said output delivering said liquid cleaner into said drain pan when said pump is turned on wherein said liquid cleaner is configured to clean and sanitize the condensation that is captured in said drain pan such that mold and algae are inhibited from growing in the condensation which could potentially obstruct a drain line of said drain pan;
a processor being positioned within said housing, said pump being in electrical communication with said pump;
an electronic clock being positioned within said housing, said electronic clock being in electrical communication with said pump, said electronic clock tracking a time of day, said electronic clock being actuatable to count down a duration of time associated with a timer function of said electronic clock, said pump being turned on when said electronic clock begins counting down said duration of time associated with said timer function, said pump being turned off when said electronic clock completes counting down said duration of time associated with said timer function;
a display being coupled to said front wall of said housing, said display being in electrical communication with said pump, said display displaying day indicia comprising days of the week, said display displaying time indicia comprising numbers to indicate said time of day, said display displaying timer function indicia comprising numbers to indicate said duration of time associated with said timer function; and
a plurality of control buttons, each control button of said plurality of control buttons being movably integrated into said housing, each control button of said plurality of control buttons being in electrical communication with said processor, said plurality of control buttons including a menu button and an up button and a down button and a select button.

2. The air conditioner cleaning device according to claim 1, wherein:
said housing has a front wall and a back wall and an outer wall extending between said front wall and said back wall;
said housing having an attachment element being disposed on said back wall;
said attachment element is attachable to said air handler;
said outer wall has a bottom side;
said input extends through said bottom side; and
said output extends through said bottom side.

3. The air conditioner cleaning device according to claim 2, wherein:
said container has a neck;
said neck is open into an interior of said container;
said device includes a lid being removably attachable to said neck of said container;
said lid has an upper wall;
said lid has a hole extending through said upper wall;
said device includes an input tube being coupled to said input of said pump; and
said input tube is extendable through said hole in said lid.

4. The air conditioner cleaning device according to claim 3, wherein:
said input tube extends downwardly into said interior of said container;
said input tube is submerged in said liquid cleaner; and
said pump urges said liquid cleaner into said input of said pump when said pump is turned on.

5. The air conditioner cleaning device according to claim 1, wherein:
said air conditioner cleaning device includes an output tube being coupled to said output of said pump;
said output tube receives said liquid cleaner when said pump is turned on;
said output tube is extendable into said air handler; and
said output tube is routed into said drain pan.

6. The air conditioner cleaning device according to claim 5, wherein said output tube releases said liquid cleaner into said drain pan when said pump is turned on wherein said liquid cleaner is configured to disinfect and clean the condensation that collects in said drain pan.

7. The air conditioner cleaning device according to claim 1, wherein:
said housing has an outer wall;
said outer wall has a top side and a bottom side; and
said display is positioned closer to said top side than said bottom side of said outer wall of said housing.

8. The air conditioner cleaning device according to claim 7, wherein:
said plurality of control buttons is positioned between said display and said bottom side of said outer wall of said housing; and
said plurality of control buttons is spaced apart from each other and is distributed along an entire width of said display.

9. The air conditioner cleaning device according to claim 1, wherein:
said display is actuated to display a menu when said menu button is depressed;
said up button moves a cursor upwardly along said menu when said menu is displayed on said display and when said up button is depressed;
said down button moves said cursor downwardly along said menu when said menu is displayed on said display and said down button is depressed; and
said cursor selects an option in said menu when said select button is depressed.

10. The air conditioner cleaning device according to claim 1, wherein:
said housing has a front wall;
said housing has word indicia being applied to said front wall of said housing;
said word indicia comprises the words “menu” and “up” and “down” and “select”;
said word “menu” is positioned beneath said menu button;
said word “up” is positioned beneath said up button;
said word “down” is positioned beneath said down button; and
said word “select” is positioned beneath said select button.

11. The air conditioner cleaning device according to claim 1, wherein:
said housing has an outer wall;
said air conditioner cleaning device includes a power cord being coupled to and extending away from said outer wall of said housing;
said power cord is in electrical communication with said processor; and
said power cord is electrically coupled to a power source comprising a power supply of said air handler wherein said power cord is configured to supply electrical power to said processor without requiring a user to replace batteries in said housing.

12. An air conditioner cleaning device comprising:
an air handler of a heating, ventilation and air conditioning system, said air handler having an evaporator coil, said evaporator coil having a drain pan wherein said drain pan is configured to capture condensation that drips from said evaporator coil;
a housing being mountable to said air handler, said housing having a front wall and a back wall and an outer wall extending between said front wall and said back wall, said housing having an attachment element being disposed on said back wall, said attachment element being attachable to said air handler;
a pump being positioned within said housing, said pump having an input and an output, said outer wall of said housing having a bottom side, said input extending through said bottom side, said output extending through said bottom side;
a container containing a liquid cleaner, said container having a neck, said neck being open into an interior of said container, said input of said pump being in fluid communication with said container, said pump urging said liquid cleaner into said input and outwardly through said output when said pump is turned on, said output being in fluid communication with said drain pan, said output delivering said liquid cleaner into said drain pan when said pump is turned on wherein said liquid cleaner is configured to clean and sanitize the condensation that is captured in said drain pan such that mold and algae are inhibited from growing in the condensation which could potentially obstruct a drain line of said drain pan;
a lid being removably attachable to said neck of said container, said lid having an upper wall, said lid having a hole extending through said upper wall;
an input tube being coupled to said input of said pump, said input tube being extendable through said hole in said lid, said input tube extending downwardly into said interior of said container, said input tube being submerged in said liquid cleaner, said pump urging said liquid cleaner into said input of said pump when said pump is turned on;
an output tube being coupled to said output of said pump, said output tube receiving said liquid cleaner when said pump is turned on, said output tube being extendable into said air handler, said output tube being routed into said drain pan, said output tube releasing said liquid cleaner into said drain pan when said pump is turned on wherein said liquid cleaner is configured to disinfect and clean the condensation that collects in said drain pan;
a processor being positioned within said housing, said pump being in electrical communication with said pump;
an electronic clock being positioned within said housing, said electronic clock being in electrical communication with said pump, said electronic clock tracking a time of day, said electronic clock being actuatable to count down a duration of time associated with a timer function, said pump being turned on when said electronic clock begins counting down said duration of time associated with said timer function, said pump being turned off when said electronic clock completes counting down said duration of time associated with said timer function;
a display being coupled to said front wall of said housing, said display being in electrical communication with said pump, said display displaying day indicia comprising days of the week, said display displaying time indicia comprising numbers to indicate said time of day, said display displaying timer function indicia comprising numbers to indicate said duration timer function associated with said timer function, said outer wall of said housing having a top side, said display being positioned closer to said top side than said bottom side of said outer wall of said housing;
a plurality of control buttons, each control button of said plurality of control buttons being movably integrated into said front wall of said housing, each control button of said plurality of control buttons being in electrical communication with said processor, said plurality of control buttons including a menu button and an up button and a down button and a select button, said plurality of control buttons being positioned between said display and said bottom side of said outer wall of said housing, said plurality of control buttons being spaced apart from each other and being distributed along an entire width of said display, said display being actuated to display a menu when said menu button is depressed, said up button moving a cursor upwardly along said menu when said menu is displayed on said display and when said up button is depressed, said down button moving said cursor downwardly along said menu when said menu is displayed on said display and said down button is depressed, said cursor selecting an option in said menu when said select button is depressed;
wherein said housing has word indicia being applied to said front wall of said housing, said word indicia comprising the words “menu” and “up” and “down” and “select”, said word “menu” being positioned beneath said menu button, said word “up” being positioned beneath said up button, said word “down” being positioned beneath said down button, said word “select” being positioned beneath said select button; and
a power cord being coupled to and extending away from said outer wall of said housing, said power cord being in electrical communication with said processor, said power cord being electrically coupled to a power source comprising a power supply of said air handler wherein said power cord is configured to supply electrical power to said processor without requiring a user to replace batteries in said housing.

13. A method of sanitizing a drain pan of an evaporator coil of a heating, ventilation and air conditioning system, the steps of the method comprising:
attaching a housing to an air handler of the heating, ventilation and air conditioning system;
wiring a power cord of the housing into a power supply of the air handler such that the power cord supplies electrical power to pump that is disposed within the housing;
inserting an input hose through a hole in a lid that is attached to a container of a liquid cleaner, said input hose being in fluid communication with an input of said pump such that said pump urges the liquid cleaner to travel through said input hose;
inserting an output hose into a drain pan of an evaporator coil of the air handler, said output hose being in fluid communication with an output of said pump such that said pump urges the liquid cleaner into the drain pan wherein said liquid cleaner is configured to sanitize condensation from said evaporator coil that collects in said drain pan; and
programming an electronic timer which is disposed within said housing to turn on said pump at a chosen time and to turn off said pump when a selected duration of time has elapsed such that said pump automatically pumps said liquid cleaner into said drain pan at said chosen time.
